(19) United States
(12) Patent Application Publication
(10) Pub. No.: US 2012/0302460 A1
(43) Pub. Date: Nov. 29, 2012

(54) Use of Low Affinity Neurotrophin Receptor P75 As Marker for High Differentiation Potential Muscle Stem Cells, Muscle Satellite Cells, And Degenerative Skeletal Muscle Diseases

(75) Inventors: Cinthia Farina, Siziano (Pavia) (IT); Emanuela Colombo, Inverigo (Como) (IT)

(73) Assignee: Fondazione I.R.C.C.S. ISTITUTO NEUROLOGICO "Carlo, Milano (IT)

(21) Appl. No.: 13/575,439

(22) PCT Filed: Oct. 13, 2010
(86) PCT No.: PCT/EP2010/065315

§ 371 Date: Jul. 26, 2012

(30) Foreign Application Priority Data
Jan. 26, 2010 (IT) MI2010A 000095

(51) Int. Cl. C07K 14/705; C12N 5/077; C40B 30/04; G01N 33/566; C12Q 1/68

(52) U.S. Cl. 506/9; 435/7.21; 435/6.12; 435/6.11; 435/375; 435/366; 530/350

(57) ABSTRACT

The marker for highly differentiating power muscle stem cells, muscle satellite cells and degenerative skeletal muscle diseases consists in the receptor recorded as PG8138, TNR16_HUMAN in the UniProtKB database, expressed in the skeletal muscle cells. The cellular expression of said receptor is also determined by the identification of highly differentiating power muscle stem cells and satellite cells.

The present invention relates to a marker for highly differentiating power muscle stem cells, muscle satellite cells and degenerative skeletal muscle diseases; an identification method for highly differentiating power muscle stem cells and muscle satellite cells; a control method for the production of muscle dystrophin; a method for the production of a non-human transgenic mammal for the study of degenerative skeletal muscle diseases and a non-human mammal thus obtained.

Neurotrophins (NT) are a family of trophic factors that play an essential role in controlling dendritic growth and the number of neurons. Nerve Growth Factor (NGF), Brain-Derived Neurotrophic Factor (BDNF), Neurotrophin 3 (NT3) and Neurotrophin 4/5 (NT4/5) all belong to this family. They carry out different functions aimed at aiding the development and maintenance of vertebrates' nervous system, where they promote various processes such as cellular survival, differentiation or apoptosis. For example, they modulate the neuronal differentiation programmes; they regulate the number of synapses and their transmission efficacy and also intervene in maintaining the nervous system's plasticity, providing suitable responses to stimuli and insults (Bibel and Barde. 2000).

Neurotrophic factors and their receptors in the neuromuscular system seem to work as modulators for the development and maintenance of motor neurons. The post-synaptic muscular fibre can release NTs that link the receptors in the presynaptic terminals of the motor neurons. They are then transferred from there, using retrograde transport, to the neuronal cell body, where they stimulate motor neuron survival (Yano and Chao. 2004). The NTs can increase the presynaptic release of neurotransmitters and are essential for maintaining the post-synaptic region in muscles (Wang and Poo. 1997, Xie, et al. 1997, Gonzalez, et al. 1999). In vitro studies and studies on animal models have suggested that these molecules do not only act on neurons but also on muscle cells. For example, NGF and BDNF seem to be able to regulate the myogenic differentiation process in rodent muscle cells in vitro. (Seidl, et al. 1998, Rende, et al. 2000, Mousavi and Jasmin. 2006). An altered expression of NTs and their receptors were found in several experimental pathology models (review (Chevrel, et al. 2006)). Their relevance in muscle physiology and human pathologies is not yet clear, however.

In particular, the invention relates to the neurotrophin receptor p75NTR. This molecule, belonging to the TNF receptors family, is able to link all the NTs with similar affinities. When activated it starts off a cascade of intracellular signal transduction, whose final response may vary depending on the cellular context in which this receptor is expressed. p75NTR is involved in both cell survival processes and in the modulation of pro-apoptotic and anti-apoptotic factors that bring about the activation of caspases and therefore cell death. It is also able to promote the stopping of the cell cycle or neuronal growth, through the inactivation of Rho-kinase (review (Dechant and Barde. 2002)).

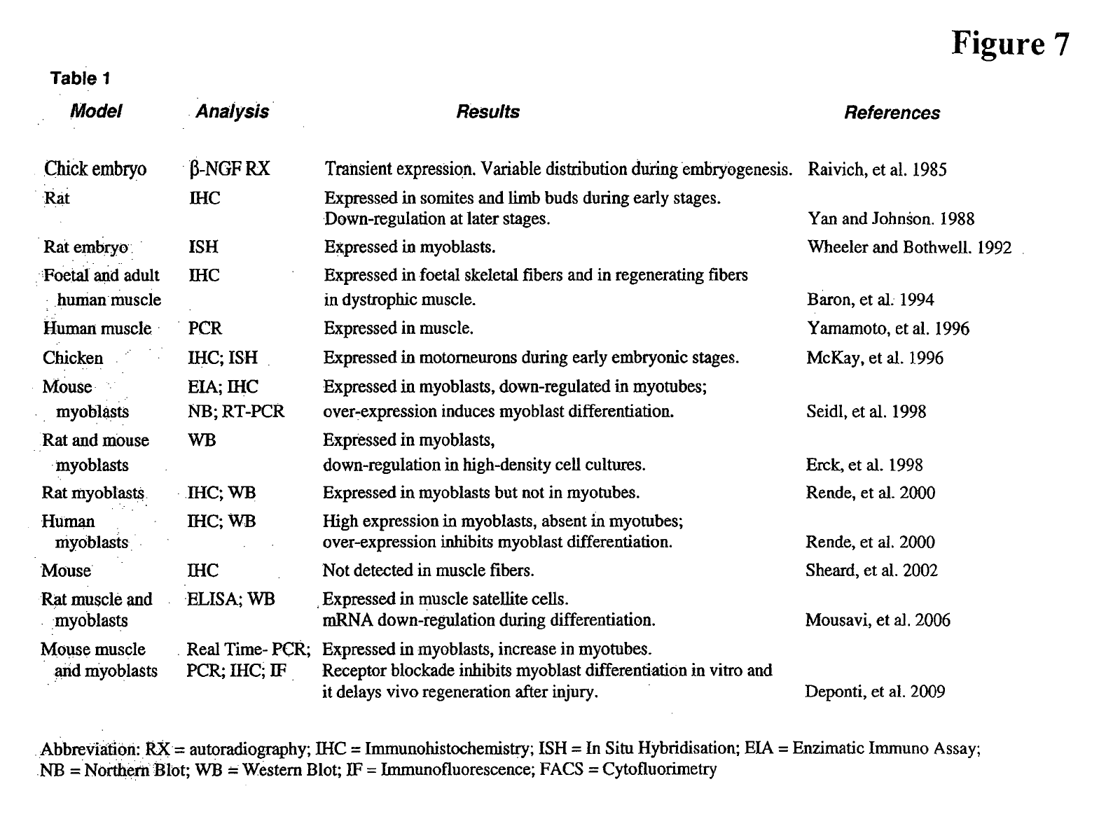

There is conflicting data about the expression of p75NTR in the skeletal muscle (table 1, FIG. 7). The technical task of the present invention was to check the involvement of the neurotrophin receptor recorded as P08138, TNR16_HUMAN (Tumor necrosis factor receptor superfamily member 16) in the nomenclature of the UniProtKB database and defined using the common name p75NTR, in muscle physiology, and in the alteration processes in muscle functionality and tissue reconstruction in order to evaluate its diagnostic/therapeutic use.

The invention discloses a marker for human or animal muscle high differentiating power stem cells, muscle or human satellite cells and for acquired or inherited human or animal degenerative skeletal muscle diseases, consisting in the receptor recorded as P08138, TNR16_HUMAN in the UniProtKB database, expressed in the human or animal skeletal muscle cells.

The invention also discloses a method for the identification of human or animal muscle high differentiating power stem cells, and human or animal muscle satellite cells, consisting in the determination of cellular expression of the above-stated receptor.

The invention also provides a cell preparation comprising human or animal muscle high differentiating power stem cells isolated from the muscle and expressing the above-stated receptor for the therapy of muscle diseases, a transport vector for the gene p75NTR(NGFR) or the protein thereof synthesised for the therapy of muscle diseases, a cell preparation comprising human or animal muscle satellite cells isolated from the muscle and expressing the above-stated receptor for therapy of muscle diseases, the engineering of human or animal muscle satellite cells isolated from the muscle for the induction of the expression of the above-stated receptor, and a method for the control, regulation or induction of the production of dystrophin in human or animal muscle via the induction or stimulation of cell expression of the above-stated receptor.

Finally the invention discloses a method for the production of a non-human transgenic mammal for the study of degenerative muscle diseases characterised by the fact that the animal's genome is modified in order to eliminate the functionality of the gene p75NTR(NGFR) in the skeletal muscle cells. The animal genome modification increases the functionality of the gene p75NTR in the skeletal muscle cells. In particular, the gene p75NTR(NGFR) tends to be over-expressed.

The non-human transgenic mammal with a genome modified in this manner is preferably a mouse.

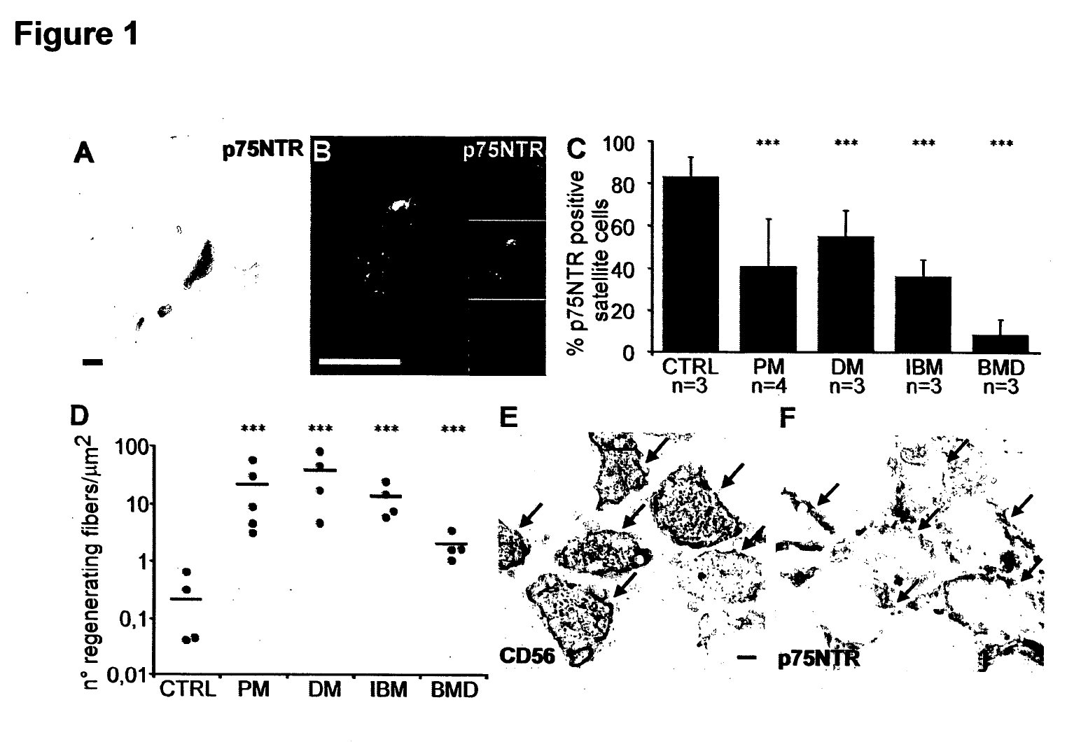

The invention is described with reference to FIGS. 1-8 attached.

The description of FIGS. 1-6 can be found in the list of keys contained below.

FIG. 7 shows a table 1 with the conflicting data on the expression of p75NTR in the skeletal muscle.

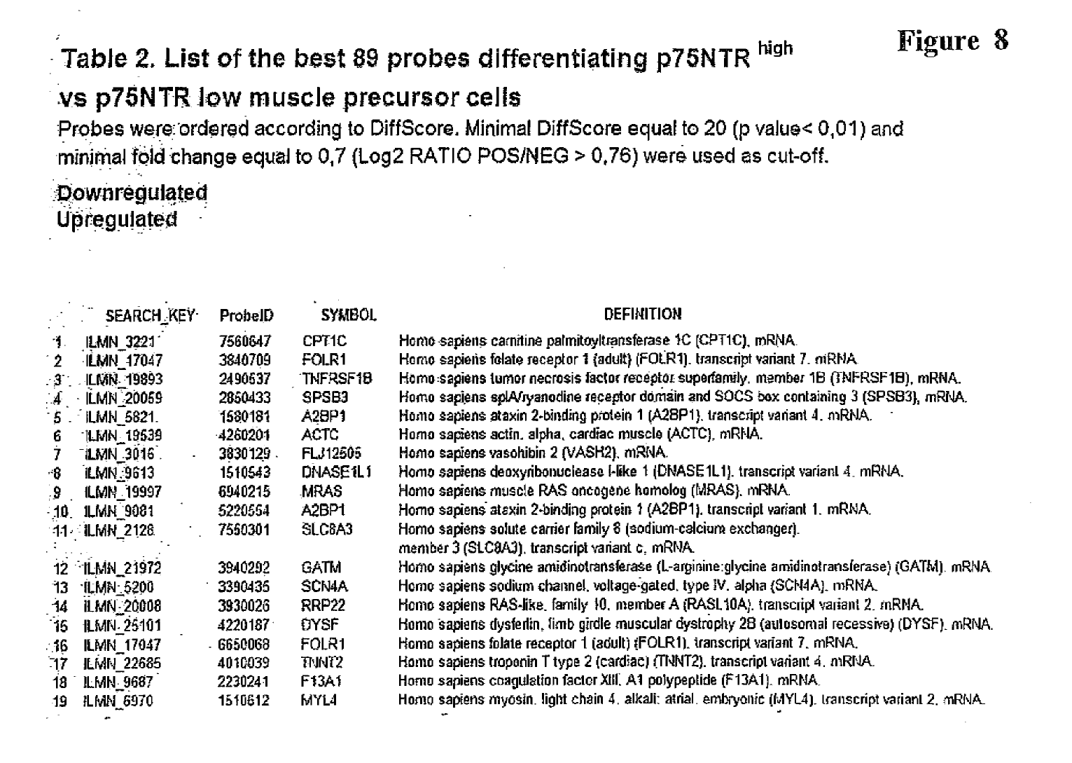

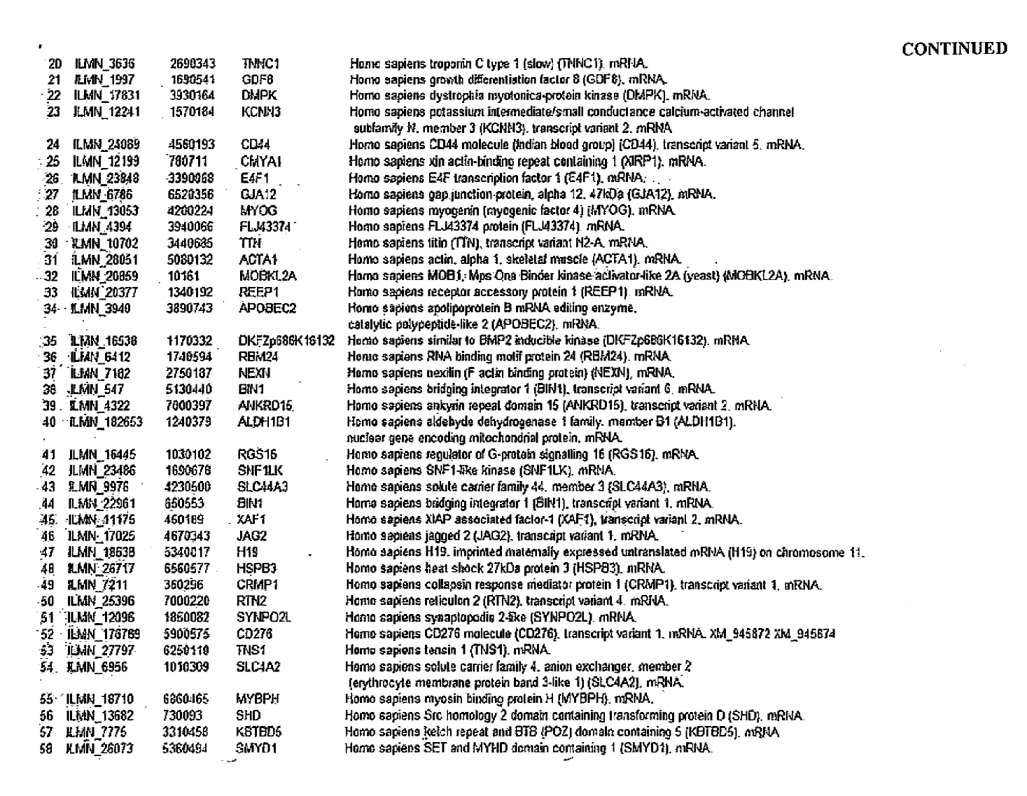

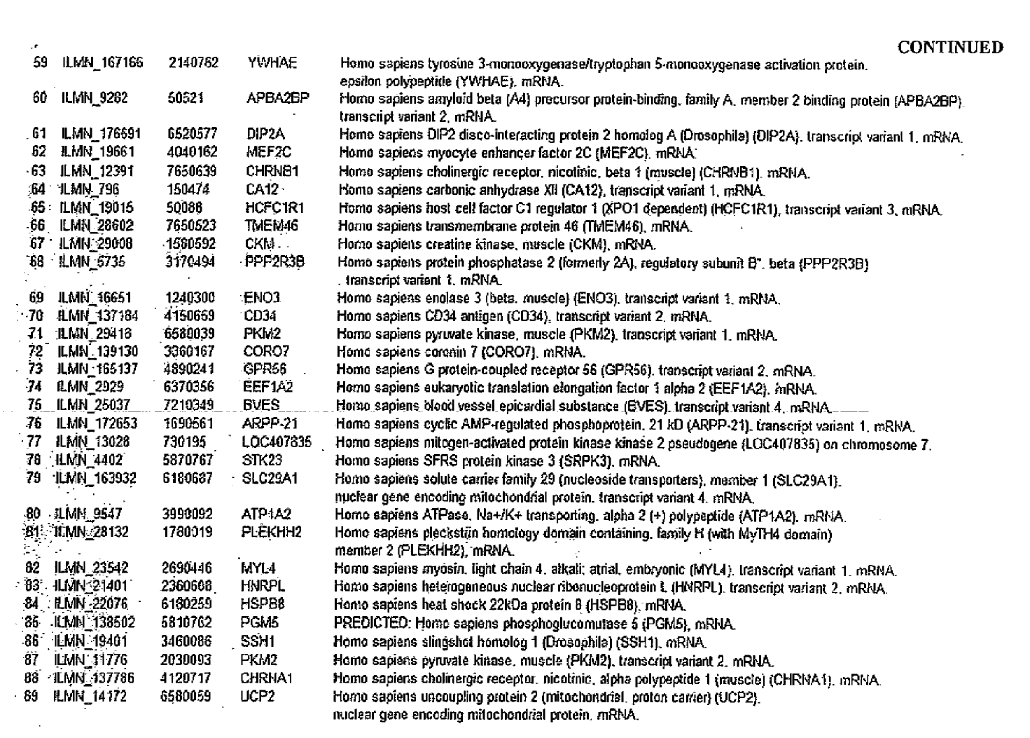

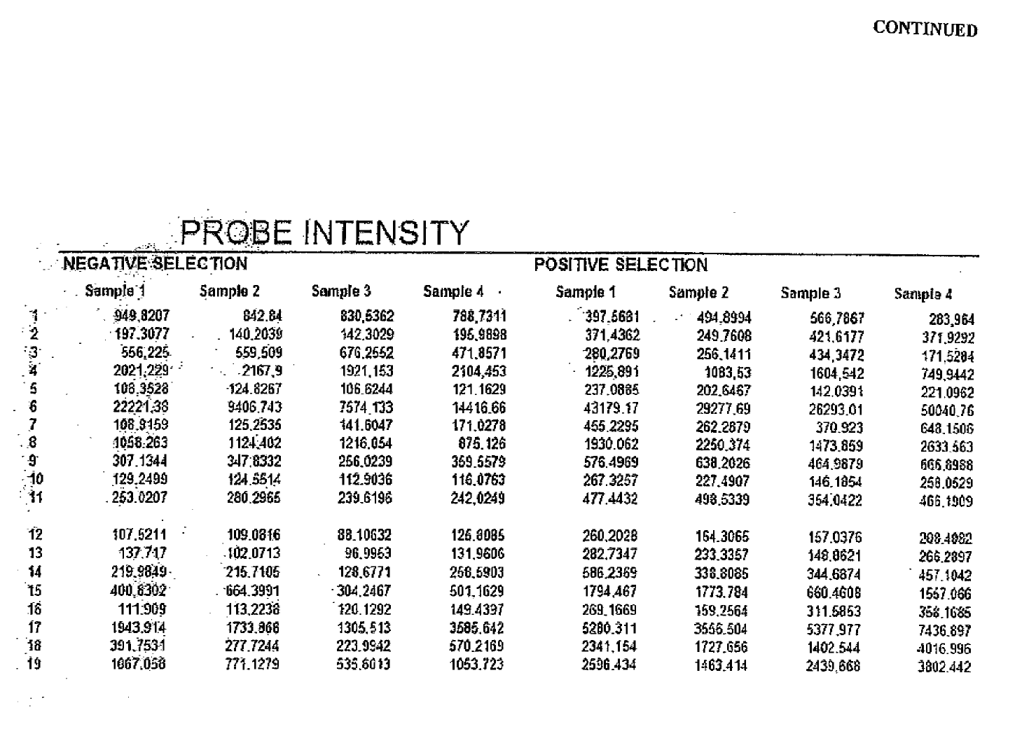

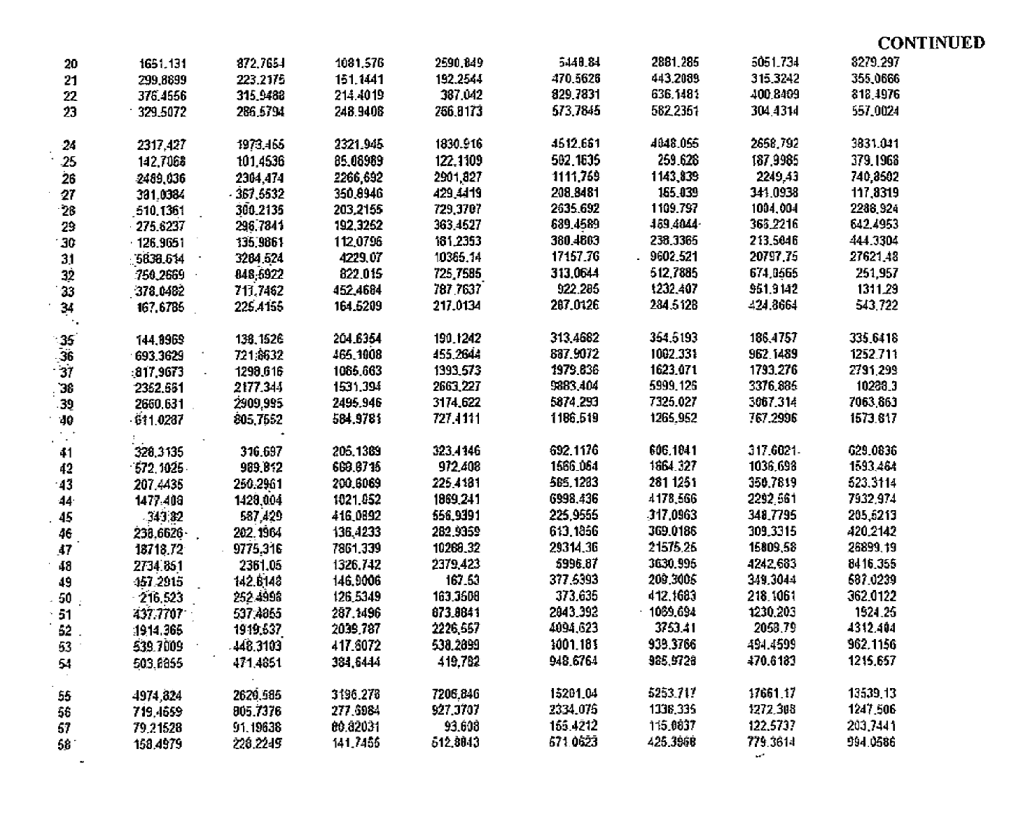

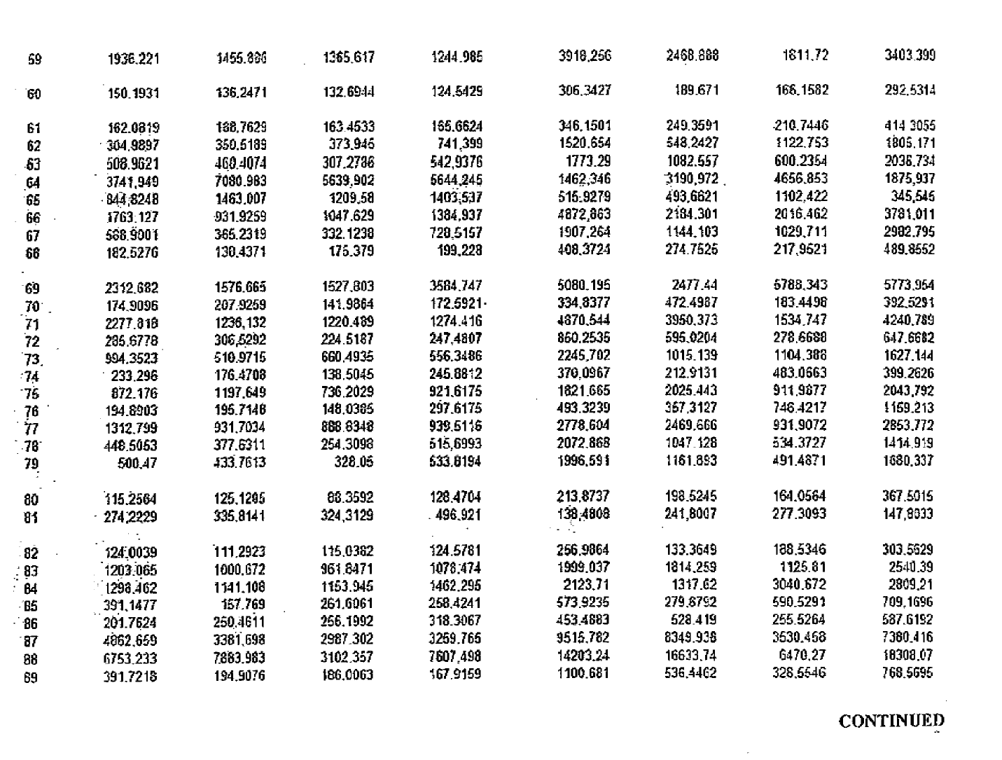

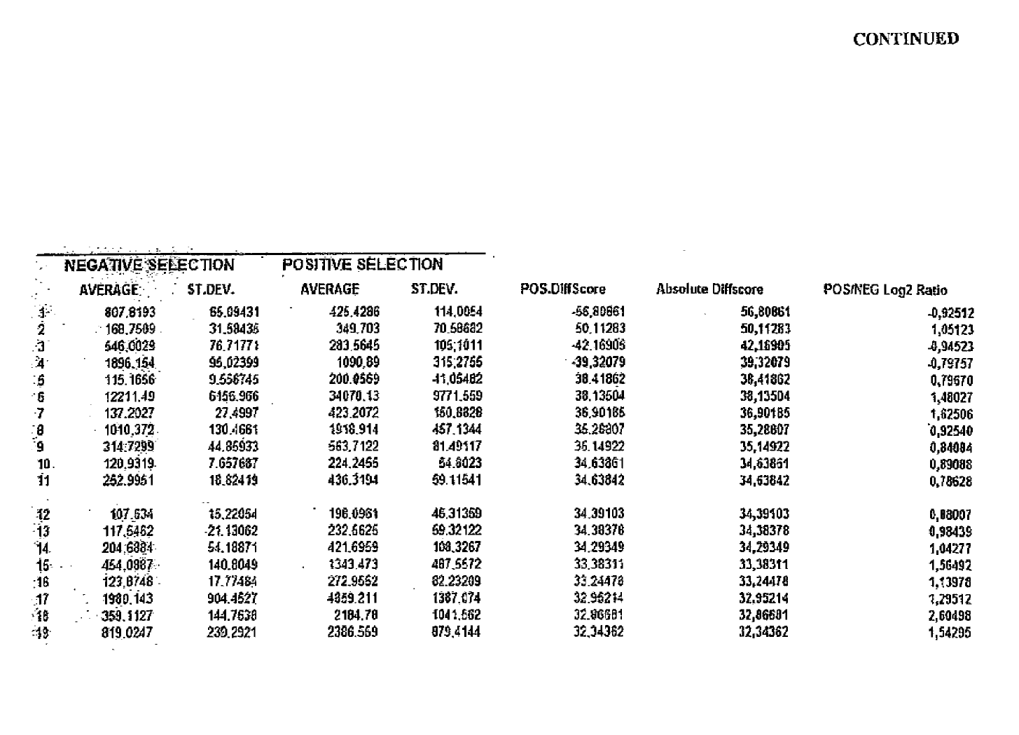

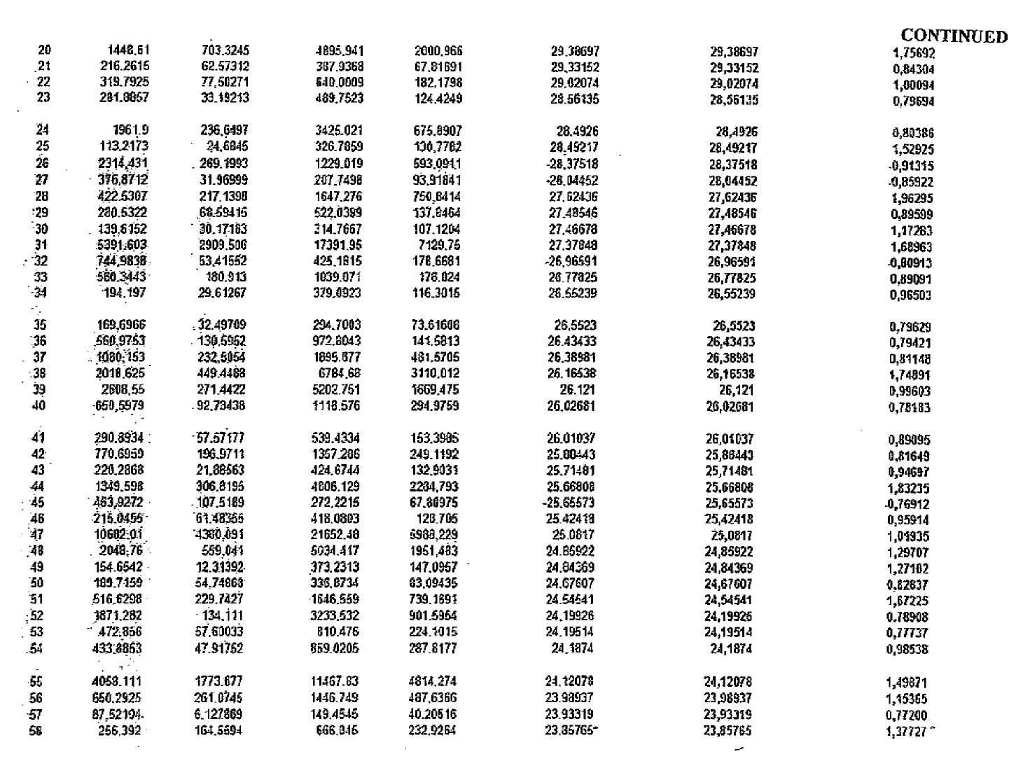

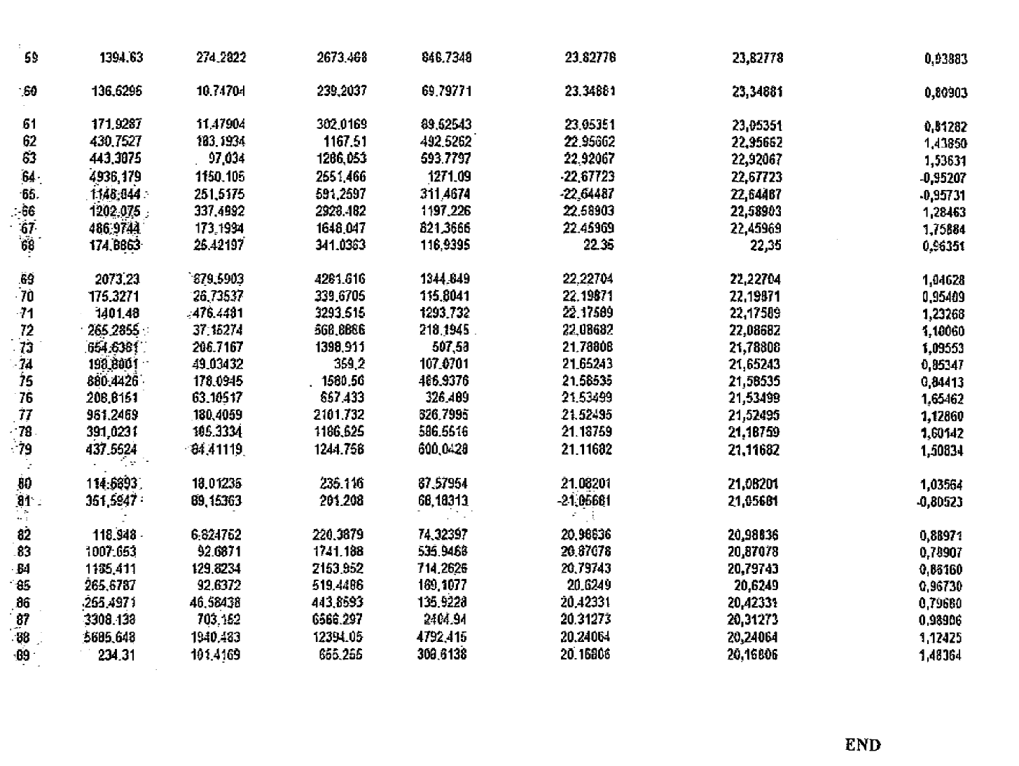

FIG. 8 shows a table 2 with the 89 gene probes that exceeded the set threshold of significance.

The invention starts with observation on the role of the receptor p75NTR in human muscle physiology and pathology. We analysed two categories of muscle diseases: inflammatory myopathies and Becker's muscular dystrophy. While the first are a group of acquired pathologies, the second is genetically-based.

Inflammatory myopathies, dermatomyositis (DM), polymyositis (PM) and inclusion-body myositis (IBM) are characterised by the onset of a progressive weakness in the skeletal muscle, associated with the massive introduction of inflammatory cells that are then positioned in the perimysium, perivascular and endomysium areas of tissue. They are significantly different from each other, from both a clinical and a physiopathological point of view. PM is an inflammatory myopathy mediated by cytotoxic T cells. DM seems to be an angiopathy mediated by antibodies, characterised by myositis and dermatitis (Dalakas and Hohlfeld. 2003). IBM is more common in people over 50 years of age; it is the most important myopathy associated with age and seems to be a degenerative disease with secondary inflammation (Needham and Mastaglia. 2007).

Becker's muscular dystrophy is a genetic disease characterised by the onset of a progressive muscle weakness and the loss of tissue integrity, and is caused by a reduction of the amount or an alteration of the size of dystrophin. Muscle in patients affected by dystrophy is characterised by the presence of necrotic and degenerating fibres.

It was found that p75NTR is a marker for satellite cells in human skeletal muscle and for regenerating fibres in the damaged muscle.

We examined the location of p75NTR in human skeletal muscle via immunohistochemistry and immunofluorescence. Muscle biopsies with normal histology were used as controls. In these tissues, the receptor is under expressed by the muscle fibres, while it is present on some cells, called satellite cells, that can be found near the fibre and which are a pool of muscle stem cells that are normally dormant but ready to be reactivated in the event of tissue damage. The co-location of p75NTR with the satellite cell marker NCAM/CD56 was confirmed under confocal microscopy (FIG. 1A-B).

Positive p75NTR satellite cells were also found in rat muscle (Mousavi and Jasmin. 2006), however the quantitative evaluation of this sub-group of satellite cells has never been carried out. In our system, the counting of satellites expressing p75NTR has demonstrated how most of them in healthy adult muscle express the neurotrophins receptor (FIG. 1C, first column), indicating p75NTR as a new marker for this cell type.

We therefore investigated whether pathological muscle conditions could alter the pool of muscle precursors expressing p75NTR. For this reason we selected and analysed samples of tissue from patients affected by inflammatory myopathy (PM, DM, IBM), where the pathogenetic process is presumably of autoimmune origin, or by BMD, where muscular degeneration is caused by a defect in the dystrophin gene. The percentage of positive p75NTR satellite cells in both the inflamed and the dystrophic muscle is significantly reduced (P<0.001, FIG. 1C), clearly indicating a deficit in this population of precursor cells in the pathological muscle.

To the contrary, the regenerative process (visible by the presence of new fibres that are still CD56/NCAM positive), which is not easily detectable in healthy human muscle, was considerable in inflammatory myopathies and in BMD (P<0.001 FIG. 1D). In accordance with a previous study that described the p75NTR on muscle regeneration in patients with Duchenne muscular dystrophy (Baron, et al. 1994), we showed that the new generation fibres also express p75NTR in inflammatory myopathies and in BMD (FIG. 1E-F), indicating a potential role for this receptor in the early cell fusion and differentiation phases.

In brief, these in vivo observations strongly imply that the p75NTR satellite cells constitute a critical group of precursor cells for tissue reconstruction.

It was found that p75NTR is expressed by human precursor muscle cells: it is downregulated in inflammatory conditions, while it is temporarily upregulated during differentiation.

We then extended the in vitro test to human primary cultures of muscle precursors, the so-called myoblasts.

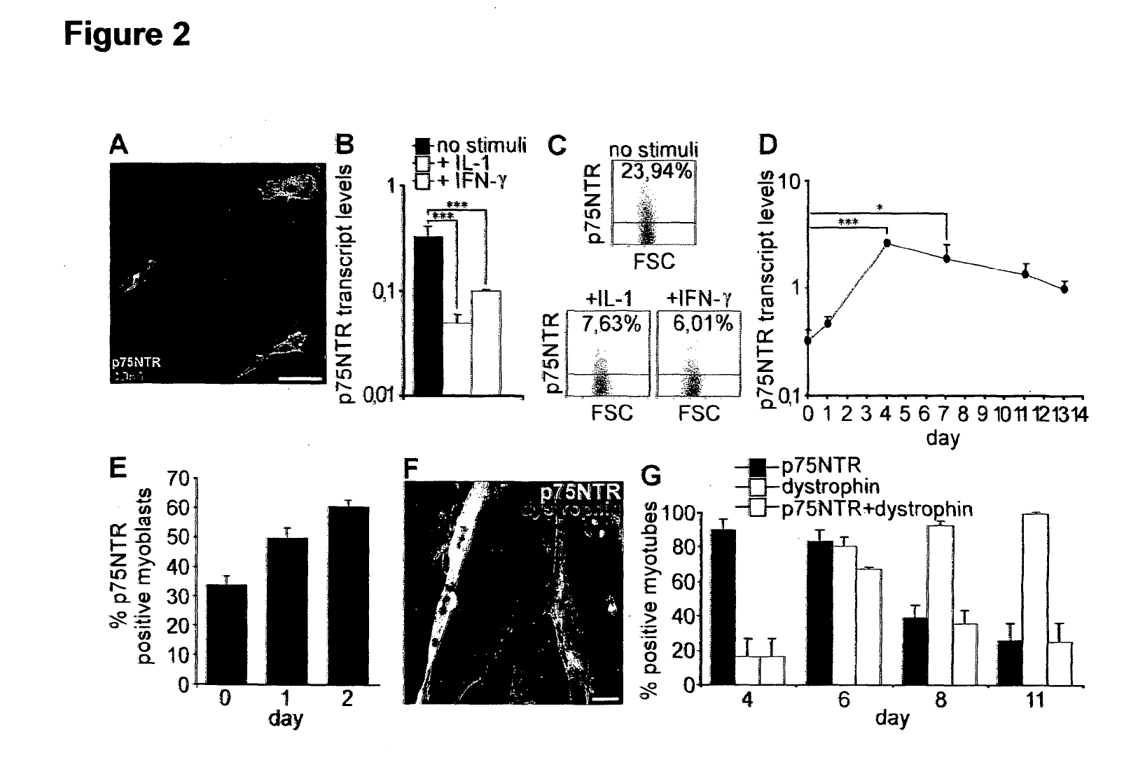

First of all, we saw that the myoblasts are able to express the receptor at basal conditions (FIG. 2A). As the percentage of satellite cells expressing p75NTR decreased in vivo in the diseased muscle, we evaluated in vitro whether the inflammatory mediators could be one of the causes of such downregulation. In fact, when the myoblasts were exposed in vitro to inflammatory cytokines such as IL-1 or IFN-γ, the mRNA and protein levels of p75NTR decreased markedly (P<0.001 FIG. 2B-C).

We then monitored expression of the receptor during myoblast fusion and in vitro differentiation into multinucleated elements, called myotubes. A couple of studies on rodent cell lines have reported the reduction in expression of p75NTR during cell differentiation (Seidl, et al. 1998, Rende, et al. 2000, Mousavi and Jasmin. 2006). In human primary cells, the levels of p75NTR transcript drastically increased during the first days of the myogenic process, and then decreased once more (FIG. 2D). The cytofluorimetric analysis showed that the differentiating stimuli were able to rapidly upregulate p75NTR on the myoblasts surface (FIG. 2E). Moreover, this molecule was expressed on the multinucleated myotubes (FIG. 2F). About 90% of the myotubes expressed p75NTR at day 4 after differentiation induction, while only 25% of the myotubes were present at day 11 (FIG. 2G, black columns). These data, which show that p75NTR is present during the early stages of differentiation and is downregulated after the in vitro maturation of the myotubes, are in accordance with the presence of the receptor on regenerating fibres and its loss of expression on mature fibres in vivo.

Finally, the expression kinetics of p75NTR and dystrophin, an essential protein for muscle, which is induced during myotube maturation, were compared in myotubes. Dystrophin was present on most myotubes at day 6 and remained also later (FIG. 2G, white columns). Worthy of note, is the fact that the dystrophin was initially only produced by myotubes that already expressed p75NTR (FIG. 2G, day 4), indicating that the presence of this receptor on multinucleated elements is prior to the appearance of dystrophin on a timescale and is probably able to regulate its expression.

It was found that p75NTR regulates differentiation of precursor cells.

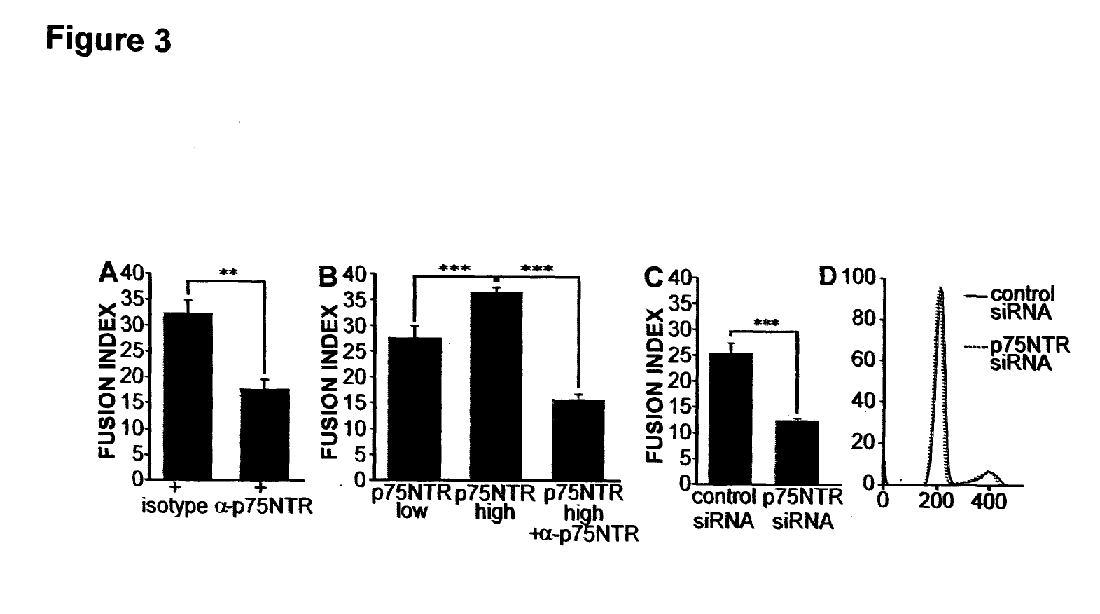

As p75NTR is present on satellite cells in vivo and in vitro, its expression increases during the myoblast fusion process and can be found in the early stages of muscle fibre regeneration, the invention wished to clarify the contribution of this molecule in muscle cell differentiation. Functional experiments carried out in vitro showed that p75NTR plays an essential role in promoting the myogenic process. We initially blocked p75NTR activity by administering an anti-p75NTR blocking antibody during culture and we examined the fusion process under these conditions. As shown in FIG. 3A, the muscle cells treated with the blocking antibody signalled lower levels of fusion index compared to the control cells (P=0.001).

We then separated the myoblasts expressing p75NTR from the negative ones for the receptor and obtained two populations with about ten times the difference in p75NTR transcript expression at the moment of differentiation induction. Under these conditions, the muscle cells p75NTR^{high} were able to form significantly more myotubes than the p75NTR^{low} population (P<0.001, FIG. 3B). As expected, the addition of the blocking antibody to the p75NTR^{high} muscle precursors prevented cellular fusion (P<0.001, FIG. 3B, third column).

Finally RNA interference for p75NTR experiments were carried out. The myoblasts were transfected with siRNA for p75NTR or with non-specific control siRNA, and were induced to differentiate after 48 hours. The myoblasts treated with siRNA for p75NTR showed a sizeable decrease in the fusion index compared to the cells treated with control siRNA (P<0.001, FIG. 3C).

Therefore, p75NTR is able to influence myogenesis either by intervening directly with a specific action on this cellular programme or by modifying several functions, including differentiation. To clarify this point, we checked the effect of p75NTR silencing on myoblast vitality and proliferation. These functions were not altered after treatment and the progression of the cellular cycle had also not been changed further to receptor silencing (FIG. 3D).

To conclude, these data show that p75NTR specifically controls the differentiation process in human stem precursors. These observations are in accordance with some studies carried out on lines of rodents in culture, in which it was proved that artificial induction of over-expression of p75NTR increased cellular fusion (Seidl, et al. 1998), while blocking it prevented myogenesis (Deponti, et al. 2009).

It was found that p75NTR identifies precursor muscle cells that are inclined towards differentiation.

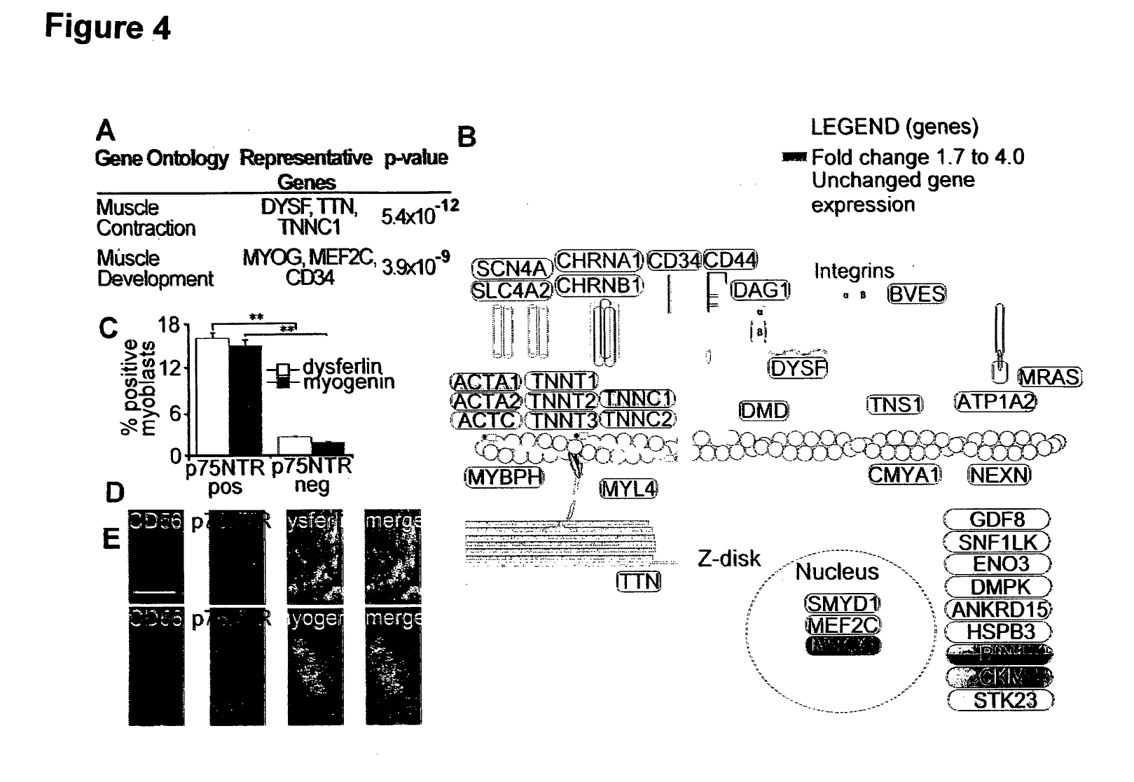

The cells expressing p75NTR were further characterised by gene profile, i.e. the transcriptome of the p75NTR^{high} cell population was compared with that of the p75NTR^{low} cells. 89 gene probes were identified, which had passed the set threshold of significance (table 2 of FIG. 8). Most of the genes were more expressed in p75NTR^{high} cells than in p75NTR^{low} cells (79 upregulated genes compared to 10 downregulated genes) and they were mostly genes highly involved in muscular processes. These genes were part of the systematic, ontological categories concerning muscular development (p<3.9×10⁻⁹) and contraction (p<5.4×10⁻¹²) (FIG. 4A). Titin, dysferlin, sub-units α and β of the nicotine receptor, actin α1, type 2 troponin T, type 1 troponin C were the most important structural genes that were found to be upregulated. We found myogenin, MEF2C, α-enolase, DMPK, CD34 of the genes assigned to muscular development.

Finally, we validated two of the upregulated genes, myogenin and dysferlin. These proteins were preferentially expressed in vitro in myoblasts expressing p75NTR (P=0.002, FIG. 4C) and their expression in positive p75NTR satellite cells was confirmed in vivo (FIG. 4D-E).

In agreement with the functional data that show how p75NTR positively regulates muscle cell fusion and differentiation, the transcriptional index shown by the positive p75NTR cells confirms that these cells have a high differentiating potential. Therefore, we propose p75NTR as a new marker for high differentiating power precursor muscle cells and we speculate that the loss of these cells in vivo in pathological situations may lead to the reduction of the tissue regeneration potential. Patients could therefore benefit from therapies aimed at reconstructing this pool of satellite cells.

It was found that p75NTR controls dystrophin induction in myotubes.

As dystrophin was only inducted on positive p75NTR myotubes, we therefore hypothesised a role for p75NTR in the maturation of muscle fibre. The involvement of p75NTR in maintenance of multinucleated muscle cells' structural integrity was investigated using gene silencing experiments.

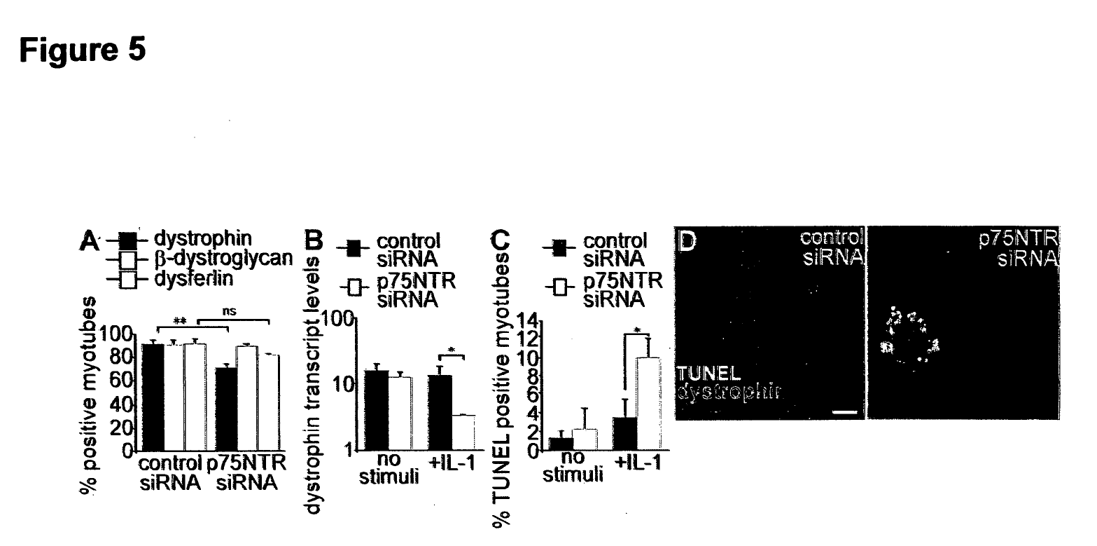

First of all, we asked ourselves whether p75NTR was necessary for dystrophin induction. The dystrophin gene was found to already be expressed in myoblasts, but the microarray test did not find any difference in the expression of this molecule among the p75NTR^{high} and p75NTR^{low} cells. The quantitative PCR for dystrophin carried out on mRNA extracted from myoblasts silenced for p75NTR or control confirmed that there is no association between p75NTR and the levels of dystrophin under base conditions. We therefore induced silenced myoblast differentiation for p75NTR and we measured the expression of dystrophin in myotubes. This test showed a significant reduction in the percentage of myotubes that express dystrophin (P=0.002, FIG. 5A), showing that p75NTR is essential for the correct expression of a structural protein like dystrophin in differentiated muscle cells. In the same way, administration of the anti-p75NTR blocking antibody during differentiation reduced the percentage of myotubes expressing dystrophin. Regulation of the dystrophin by p75NTR was specific, in fact the expression of myotubes of other muscle proteins such as dysferlin and β-dystroglycan was not altered after silencing of p75NTR (FIG. 5A). This is the first description of a cell activation pathway that is able to regulate dystrophin expression.

It was found that inflammation increases the expression of p75NTR on mature myofibres in vivo and in vitro.

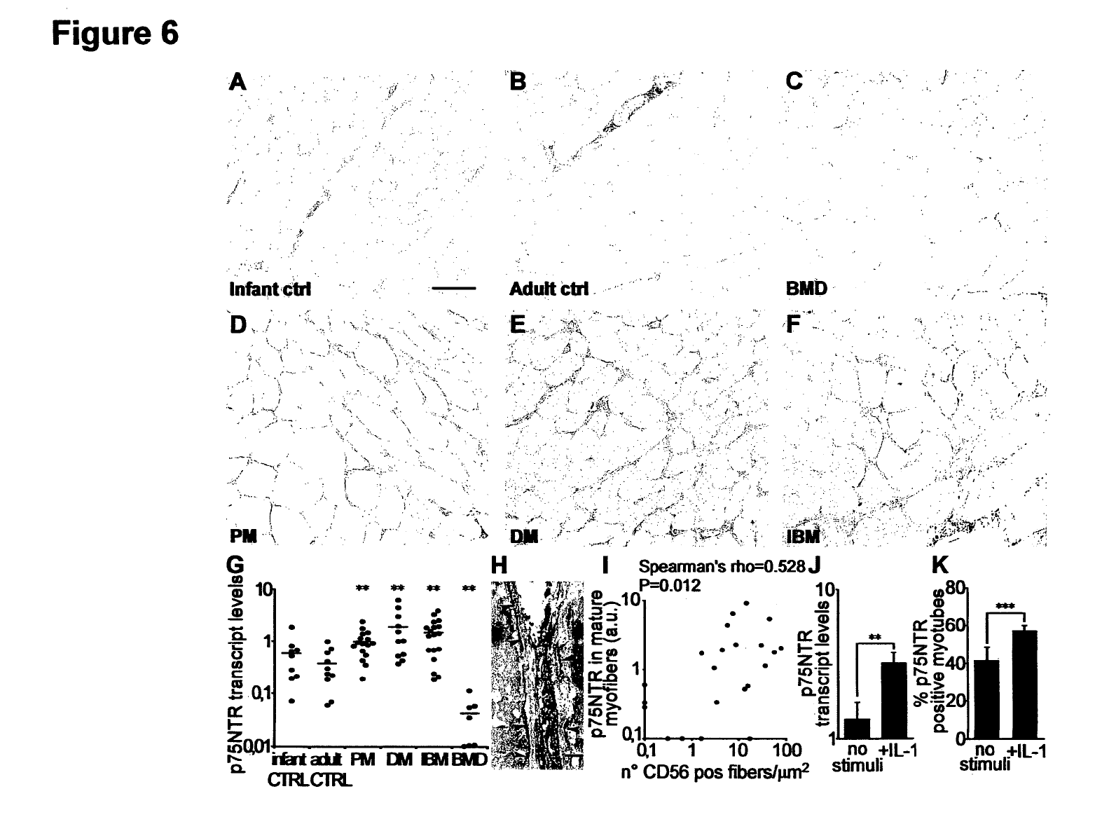

Finally, p75NTR is also involved in inflammatory muscular processes. Analysis of total receptor mRNA levels in various pathological groups showed an increase in expression of this molecule in PM, DM and IBM and, to the opposite, a clear reduction in BMD tissues (P=0.003 for PM, DM, IBM, P=0.001 for BMD compared to the adult control group FIG. 6G). Immunohistochemistry also showed an increase in immunoreactivity for p75NTR in inflammatory myopathies but not in BMD samples (FIG. 6A-F)). This marking was located in the perimysium, the endomysium and the skeletal muscle fibres in the cytoplasm and on the membrane (FIG. 6D-F, H). We measured immunoreactivity in mature fibres for p75NTR (regenerating fibres were not taken into consideration) and we noted a clear correlation between the degree of tissue regeneration and the p75NTR signal in the myofibres: indeed, samples that showed the highest levels of regeneration showed an increase of protein expression in the neurotrophin receptor on the mature myofibres, while tissues not largely regenerating (for example non-myopathic muscles) had little or no p75NTR (P=0.012, FIG. 6I). As the regeneration process was more marked in inflamed tissues, we asked ourselves whether the increase in p75NTR expression in mature myofibres could be caused by the inflammation itself. For this reason we evaluated in vitro whether exposure of the myotubes to inflammatory stimuli was able to mediate the increase in p75NTR levels. In fact, the transcript and protein levels of p75NTR significantly increased after stimulation with IL-1 (P=0.009 and <0.001, for transcript and protein respectively, FIG. 6J-K).

It was found that the p75NTR controls the myofibres' resistance to inflammatory stress.

To clarify the role of p75NTR in myofibres under conditions of stress, we silenced p75NTR in differentiated myotubes and we induced transcription of p75NTR by exposing the cells to IL-1. As shown in FIG. 5B, the block of p75NTR only causes a loss of mRNA in the dystrophin in the myotubes exposed to IL-1 (P=0.02). Moreover, under these conditions, an increase in apoptotic nuclei was shown (P=0.02, FIG. 5C-D), using TUNEL assay, therefore proving a direct role for p75NTR in the survival of myofibres under inflammatory stress conditions.

### MATERIALS AND METHODS

### Patients and Tissues

Muscle biopsies were carried out for diagnostic reasons and preserved in the Institute's tissue bank. In all cases, informed consent was obtained for the biopsy and for its use for research purposes. The tissue samples were frozen and stored in liquid nitrogen. In most cases, the biopsies were harvested from the femoral quadriceps muscle.

We selected samples with an evident diagnosis, based on clinical electromyographical and histological proof. 45 muscle biopsies from patients affected by idiopathic inflammatory myopathies were included in the study: 16 patients with PM, 11 with DM, 18 with IBM. In all cases, the histological characteristics included degeneration of myofibres, regeneration and necrosis, the presence of primary inflammation in the endomysium consisting in mononucleated cells that surrounded and/or invaded the myofibres was evident in the PM; the cases of DM were characterised by perifascicular atrophy and perivascular inflammation sometimes associated with endomysial inflammation; muscle fibres with vacuoles and endomysial inflammation were found in the cases of IBM. Treatment with immunosuppressive drugs was verified at the time of the biopsy in 3 PM, 2 DM and 3 IBM. In addition, biopsies from 7 patients affected by Becker's Muscular Dystrophy (BMD) were selected that did not show signs of immunitary infiltrates. The muscle tissue in all cases of BMD showed alterations in the expression of dystrophin and a reduction in the amount of the protein or its molecular weight were also disclosed, using western blot. Finally, biopsies from 9 children and 10 adults with no evidence of muscle diseases were included as control biopsies.

### In Vitro Culture of Myoblasts

Primary cell lines of human skeletal myoblasts were generated from muscle biopsies in patients with no sign of diseases, and were stored by the Telethon human myoblasts bank. The myoblasts were isolated by magnetic separation in order to obtain a population of pure muscle cells from the muscle biopsy. The cells are detached using trypsin (Celbio), centrifuged at 626 g for seven minutes and then re-suspended, after removing the supernatant in 1 ml of PBS+0.5% bovine serum albumin (BSA) (Calbiochem). A cell count is then carried out in order to be able to add the correct volume of the reagents required for all the cells to be separated. The cells are then centrifuged again, the supernatant is removed and the cells are incubated with anti-CD56 (BD Biosciences) antibody diluted in 1 ml of PBS+0.5% BSA at the desired concentration, for 30 minutes at 4° C. in the dark while being slowly stirred. After 2 washes, the cells are incubated with the magnetic MicroBeads (Miltenyi Biotec) diluted in PBS+0.5% BSA for 15 minutes, at 4° C., in the dark while being slowly stirred. The cells are centrifuged and are re-suspended in 500 μl of PBS+0.5% BSA. The separation column is attached to the magnet and is balanced with 3 ml of the same buffer, and after transferring the cells to the column, 3 washes are performed in order to elute the non-marked cells that are not attracted by the magnet as they do not possess beads. The column is then detached and the desired cells are then recovered by washing with 5 ml of buffer. The purity of the myoblasts preparation is verified by cytofluorimetric, which must be greater than 95%.

The myoblasts are grown in medium made from Dulbecco's modified Eagle Medium-DMEM (Euroclone), containing 20% foetal bovine serum (PAA), 100 U/ml penicillin, 100 mg/L streptomycin, 292 ng/ml L-glutamine (Euroclone), 100 μg/ml insulin (Sigma), 25 ng/ml FGF (Peprotech), 10 ng/ml EGF (Invitrogen).

The myoblasts were induced to differentiate in medium containing 2% of horse serum (M-Medical).

Myoblasts are added to IL-1 culture medium for the treatment of cytokines, at a final concentration of 100 ng/ml (R&D Systems) or to IFN-γ at a final concentration of 150 U/ml (Roche diagnostics) for 18 or 42 hours for mRNA or cytofluorimetric analysis, respectively. The mature myotubes were treated instead, for 24 or 72 hours for mRNA or protein analysis, respectively.

### Immunohistochemistry or Dual Immunofluorescence

12 control biopsies of healthy muscle (2 from children and 10 from adults), 6 PM, 7 DM, 7 IBM and 4 BMD were analysed for the immunohistochemical and immunofluorescence tests. The tissues were cut into 6 μm sections and mounted on SuperFrost Plus Microscope (Menzel-Glaeser) slides. The sections were then fixed in 50% methanol in water for 1 minute and immediately afterwards in 100% methanol for 1 minute. For immunohistochemical marking, we carried out incubation for 10 minutes with 1.5% H₂O₂ in methanol, after 3 washes in PBS, in order to block peroxidase endogenous activity and then another 3 washes in PBS. The section were marked with a water-repellent pen (DakoCytomation) and left to incubate in PBS+2% BSA with 5% of goat serum added (DakoCytomation) for 1 hour in a humid chamber. After removing the blocking solution, incubation in the humid chamber at 4° C. o/n is then carried out with the appropriate primary antibody, suitably diluted in PBS+2% BSA and previously centrifuged at 12000 rpm for 5 minutes to allow depositing of any impurities.

The following primary antibodies were used: monoclonal mouse anti-human NGFR (R&D Systems), monoclonal mouse anti-human CD56 (BD Biosciences), polyclonal rabbit anti-human dystrophin (supplied by Dr. Mora), monoclonal mouse anti-human dysferlin and anti-human β-dystroglycan (Novocastra), monoclonal mouse anti-human myogenin (DakoCytomation), purified mouse IgG1 isotype (Sigma), polyclonal rabbit Ig (DakoCytomation).

After 3 washes in PBS, the sections were then incubated with the secondary antibody at room temperature in the humid chamber, and were then washed another 3 times in PBS; for immunohistochemistry, the secondary antibody Labelled Polymer-HRP anti-mouse Ig (Ig; Envision™ system, Dako) was used and marking was disclosed with the use of the chromogenic substrate DAB (3,3′-diaminobenzidine, DakoCytomation). The sections were also marked for 5 minutes at room temperature with haematoxylin to allow the cell nuclei to be seen. The sections were then washed in H₂O to allow the haematoxylin colour to change.

The following secondary antibodies were used for immunofluorescence: Alexa 488-conjugated donkey anti-mouse IgG and Alexa 594-conjugated donkey anti-rabbit IgG (Invitrogen). The sections were also marked for 10 minutes at room temperature with Dapi, a specific colouring agent for nuclei (Sigma). Marking in immunohistochemistry was carried out by carrying out dehydration using 80% ethanol in water, 90% in water and 100% for 2 minutes each, then with 50% Bioclear in ethanol and 100% Bioclear. The preparation is covered with item-cover slides mounted on FluorSave (Calbiochem) mounting agent. In order to carry out triple marking in immunofluorescence with non-marked primary antibodies generated in the same species, it is possible to treat one of the antibodies with the Zenon kit (Invitrogen), that includes fluorescent fragments of Fab anti-mouse Ig. The dual immunofluorescence protocol is then carried out; the o/n tissue is incubated with a sole primary antibody; hybridisation is then carried out using the secondary antibody and incubation is carried out using the non-marked isotype. In the meanwhile, marking of the other antibodies using Zenon is prepared: for 1 μg of antibody, it is taken to a volume of 10 μl with PBS, 5 μl of Zenon A solution is added and left for 5 minutes at room temperature. 5 μl of Zenon B solution are then added and incubated for 5 minutes at room temperature. The desired volume is then obtained with PBS+0.2% Triton X100. The antibody is centrifuged at 12000 rpm for 10 minutes and then incubated for one hour at room temperature in a humid chamber. After washing in PBS, it is then fixed in 4% paraformaldehyde in PBS for 15 minutes. The slides are then washed again and closed using item-cover slides mounted with fluorsave. For immunofluorescence on adhering cells, the myoblasts were grown on slides in Permanox and marked using the marking protocol described above. To evaluate the presence of apoptotic nuclei, the myotubes were marked using DeadEnd™ Fluorometric TUNEL System (Promega). The myoblasts were induced for differentiation in slides with 4 wells. Once the treatment with IL-1 and siRNA was carried out, they were fixed with methanol and permeabilized with PBS+Triton at 0.2% for 15 minutes. After 3 washes in PBS, they were incubated in Equilibration Buffer for 15 minutes and then with Incubation Buffer containing nucleotides and enzyme for marking the fragmented DNA ends for 1 hour at 37° C. The reaction is then blocked by adding an equal volume of SSC 2× to the incubation buffer for 10 minutes. The slides are then washed in PBS, marked with Dapi and mounted with Fluorsave. The fluorescence images were acquired by a laser-scanning confocal microscope (Nikon), with EZ-C1 software (Nikon). The software ImageProPlus (Media Cybernetics) was used to analyse the images.

### Cytofluorimetry

The following monoclonal primary antibodies were used: mouse anti-human NGFR, mouse anti-human CD56 and IgG1 isotype (BD Biosciences) and were disclosed with the secondary PE-labelled antibody F(ab′)₂ fragments goat anti-mouse Ig (DakoCytomation).

The cells are detached from the flask by incubation with trypsin at 37° C. for 15 minutes, and centrifuged at 14000 rpm at 4° C. for 7 minutes. After decanting the supernatant, the cells are re-suspended in 1 ml of PBS+2% FCS (Facs buffer) and are then counted. 30000 cells re-suspended in 200 μl of Facs Buffer are deposited in each plate well and are centrifuged at 1400 rpm for 5 minutes at 4° C. The primary antibody diluted with Facs buffer at a volume of 50 μl is added to the cellular pellet and is left at 4° C. for 20 minutes in the dark. The pellet is then washed twice by centrifuging at 1400 rpm for 5 minutes at 4° C. with 200 μl of Facs buffer. The directly marked secondary antibody diluted in PBS+2% FCS in a volume of 50 μl is then incubated at 4° C. for 20 minutes in the dark. It is then washed again. The samples are re-suspended in 300 μl of PBS+2% FCS, transferred into the Falcon and acquired by the cytofluorimeter.

To evaluate the cell cycle progression, the cells were detached with trypsin, fixed in 70% ethanol in water, incubated overnight at 4° C., and finally marked with a solution of Propidium Iodide (PI) (50 μg/ml PI (Sigma), 0.1 mg/ml RNase A (Ambion), PBS-Triton X-100 0.05%) for 1 hour at 37° C.

The software CellQuest (BD Biosciences) and FlowJo (Tree Star Inc) were used for acquisition for data analysis.

### Treatment with Anti-p75NTR Blocking Antibody

The myoblasts were plated on 4-wells slides and induced to differentiate in medium with 10 μg/ml of monoclonal blocking antibody mouse anti-human p75NTR (Invitrogen) or mouse monoclonal Ig isotype (BD Biosciences) added. The next day, a second dose of antibody was administered. At day 6 after differentiation induction, immunofluorescence was carried out to view the individual myotubes and nuclei. The fusion index is then calculated, considering cells with more than 2 nuclei as myotubes. The calculation is carried out as follows:

FUSION INDEX=n nuclei of myotubes/n total nuclei

### Selection of Myoblasts for Expression of p75NTR

The myoblasts were separated magnetically using anti-mouse IgG microbeads (Miltenyi Biotec) after incubation with anti-human p75NTR monoclonal antibody (BD Biosciences). Both cell fractions obtained (positive and negative) were then collected and induced to differentiate. The level of transcript for p75NTR monitored via Real-Time PCR was about 10 times different between the two preparations. The selection was repeated four times on the same cell line, obtaining preparations with a similar purity. Part of the positive population was also induced to differentiate in the presence of the blocking antibody.

### p75NTR RNA Interference

The Small interfering RNA (siRNA) specific for p75NTR and the non-specific control (containing 47% GC) were purchased at Eurofins MWG. Preliminary experiments were carried out to determine the optimal concentration for silencing. The siRNA were diluted at 20 nM. Transfection was carried out via Interferin (Polyplus). The siRNA are re-suspended in Optimem (Invitrogen) at the required concentration, the transfecting agent is then added to the mix and then added directly to the culture medium after incubation at room temperature for 10 minutes. Efficacy of silencing in the various experiments was verified at day 7-10 by monitoring with quantitative PCR and ranged from 70% to 90%. Immunofluorescence for p75NTR showed a reduction in protein expression of at least 50% in the myotubes after gene silencing. Similar results were obtained with a second siRNA for p75NTR. For the silencing experiments in the myoblasts, differentiation was induced two days after transfection and myotubes analysis was carried out at day 7-10. For the experiments on the myotubes, siRNA and IL-1α were administered the same day and the cultures were analysed at day 2-4.

### RNA Extraction, cDNA Synthesis and Real-Time PCR

The total RNA was extracted using TriReagent (Ambion) from the myoblasts/myotubes in culture or from frozen muscle tissue previously homogenised with a potter. The cellular lysate was recovered and after incubation at room temperature for 5 minutes, 100 μl of chloroform per ml of TriReagent used were added to the samples. The mix was stirred vigorously for 15 seconds and then the samples were left at room temperature for 5 minutes. The first centrifugation was then carried out at 12000 g at 4° C. for 15 minutes; the mix separates into two phases: an organic phase and an aqueous phase, containing the RNA. The latter is then collected and transferred to a new Eppendorf where 250 μl of isopropanol are added. The mix is stirred on the vortex, left at room temperature for 5 minutes and then centrifuged at 12000 g at 4° C. for 10 minutes. The supernatant is decanted and the pellet is re-suspended in 500 μl of 75% ethanol; the mix is then centrifuged again at 10000 g at 4° C. for 10 minutes, the supernatant is decanted and the RNA pellet is left to dry at room temperature. Finally, the samples are re-suspended in H₂O RNAsi-Free DEPC (Ambion).

The samples are then back-transcripted to cDNA. 1 μl of random primers (Invitrogen), 1 μl of dNTP Mix 10 mM (Invitrogen), 10 pg-5 μg of total RNA are added in an RNAsi-Free Eppendorf and the final volume is taken to 14 μl with RNAsi-Free H₂O. The sample is incubated at 65° C. for 5 minutes and then placed in ice for at least 1 minute and finally centrifuged. 4 μl of First-Strand Buffer 5×, 1 μl of DTT 0.1 M and 1 μl of SuperScript III RT (200 units/μl) (Invitrogen) are added to the test-tube. The mix is mixed gently with the pipette and left at room temperature for 5 minutes. The samples are then incubated at 50° C. for 60 minutes for back-transcription and then at 70° C. for 15 minutes to deactivate the reaction.

Finally, a standard amplification in Real-Time PCR is carried out. The cDNA samples are diluted in RNAsi-Free H₂O, and the PCR reaction is prepared with 12.5 μl of Master Mix (Applied Biosystems), 1.25 μl of specific primer, 50 ng of cDNA and is taken to a final volume of 25 μl with RNasi-Free H₂O. We used the following primers: Cyclophilin A (PPIA) and dystrophin (DMD) (Applied Biosystems), p75NTR: Fw: 5′-TGTGCGAGGACACCGAGC-3′, Rw: 5′-GGGTGTGGACCGTGTAATCC-3′. Probe: 5′FAM-TGCGAGGAGATCCCTGGCCGT-3′BHQ1 (synthesised by NBS Biotech (Milan, Italy)).

A threshold of 0.1 was set for data analysis and the expression of Cyclophilin A housekeeping gene was evaluated for all samples, being found to be constant in all experimental conditions. The data obtained from the analysis of target sequences was processed using a comparison method between the Ct and the Ct of the reference gene, where Ct stands for the amplification cycle on which a significant fluorescence value is recorded in the exponential phase of the reaction.

ΔCt=Ct_{(target)}−Ct_{(reference gene)}

The expression level of the target sequences is indicated as a percentage expression compared to Cyclophilin A.

targetA/targetB=100*2^{-ΔCt}

### Microarray Analysis

The total RNA extracted from p75NTR^{high} and p75NTR^{low} myoblasts (4 independent samples for each group) was used for microarray experiments on Illumina Human_Ref-8_V3 arrays. Quantification and quality controls of the RNA were analysed using a Bioanalyzer 2100 (Agilent). Back-transcription and synthesis of cRNA biotynilate were carried out using Illumina TotalPrep RNA Amplification Kit (Ambion), in accordance with the supplier's protocol. cRNAs hybridisation was carried out on Illumina Human_Ref-8_V3 arrays (Illumina). These arrays contain about 24000 probes for exploring the transcripts contained in the Refseq database. Hybridisation of the arrays, washes, marking and scanning with Beadstation 500 (Illumina) were carried out in agreement with the supplier's protocol. The software BeadStudio (Illumina) was used to analyse the raw data grouped together by experiment condition. After cubic spline normalisation, the genes were filtered (Detection=1 in at least one experimental group) and selected according to the levels of statistically significant differential expression levels, using the Illumina custom test. The following stringency criteria were applied: minimum increase of 1.7 and p value ≦0.01 (Differential Score ≧20). Only 89 probes passed the selection. The Gene Ontology analysis was then carried out using DAVID (Dennis, et al. 2003). The Bio-information technologists did not know the type of cell being analysed. The graphic reconstruction of the data from the transcriptome analysis brought about the processing of an interactive instrument for the muscle, that links the expression data obtained in the muscle cell context. The pathway was created using Pathvisio1.1 with plug-ins (van Iersel, et al. 2008), a specific programme for biological pathway construction.

### Electronic Microscopy

12 μm sections of tissue were mounted on plastic slides for the ultra-structural marking of p75NTR. They were then fixed in PBS 4% paraformaldehyde 0.05% glutaraldehyde for 30 minutes and marked for p75NTR as in immunohistochemistry. The sections were then washed in PBS, fixed in OsO₄, dehydrated in ethanol and infiltrated in Spurr resin. Fixing in resin was achieved by turning the slides upside down on the plastic cover filled with resin. After polymerisation, the slides were removed and the resin block with the tissue section was cut and sectioned. The ultra-thin sections, both marked and non-marked with uranyl acetate and lead citrate, were examined under a Philips EM410 electronic microscope.

### Statistical Analysis

Distribution normality was verified by the Kolmogorov-Smirnov statistical analysis and, where necessary, the logarithmic transformation of data was applied. To compare significance values, the ANOVA test (for normal distribution) or the non-parametric Mann-Whitney U test (for non-normal distribution) were used. The T-test on paired samples was used to compare the values at different time points. Spearman's rho was used to evaluate correlation between the number of regenerating fibres/area and the intensity of p75NTR. All the P-values were considered at a significance level of 0.05.

### List of Keys

FIG. 1. p75NTR is a marker for human satellite cells and for regenerating fibres.

(A) Immunoreactivity for p75NTR in a satellite cell in the adult skeletal muscle. (B) Confocal image of p75NTR and CD56/NCAM. (C) percentage of satellite cells expressing p75NTR in a healthy and diseased adult muscle. (D) Quantification of CD56 positive regenerating fibres. The black bars indicate the average values for each group, each circle represents a separate sample. Expression of p75NTR (F) on CD56 positive regenerating fibres (E). Enlargement scale 5 μm in A and B, 10 μm in E and F. ***P<0.001 comparing each sample to the control group.

FIG. 2. p75NTR is expressed in in vitro myoblasts and modulated by inflammatory stimuli. It precedes dystrophin expression in differentiated cells.

(A) Dual immunofluorescence for p75NTR and CD56 in in vitro myoblasts. Down-regulation of transcript (B) and protein (C) level of p75NTR in myoblasts exposed to inflammatory stimuli for 18 and 42 hours, respectively. (D) Regulation of p75NTR transcript level during differentiation. (E) Induction of p75NTR protein levels in myoblasts exposed to differentiating stimuli evaluated by cytofluorimetry. (F) Dual immunofluorescence for p75NTR and dystrophin in mature myotubes. (G) Correlation between the expression of p75NTR in myotubes and the induction of dystrophin. Enlargement scale 30 μm in A and F. The experiments shown were carried out in triplicate and repeated at least three times in at least two primary cell lines. *P<0.05, ***P<0.001.

FIG. 3. p75NTR regulates myogenesis.

(A) Fusion index in myoblasts induced to differentiate in the presence of an anti-p75NTR blocking antibody or with isotype control. (B) Trend of myogenesis in p75NTR^{low}, p75NTR^{high}, and p75NTR^{high} myoblasts treated with anti-p75NTR blocking antibody. (C) Silencing of p75NTR in myoblasts and effect on myogenesis. Differentiation was induced two days after silencing, the myotubes were analysed at day 7-10. The experiments presented were carried out in triplicate and repeated at least three times. A and C were confirmed in two primary cell lines. **P<0.01, ***P<0.001.

FIG. 4. p75NTR identifies precursor muscle cells inclined towards differentiation.

(A) Categories of Gene Ontology significantly over-expressed in p75NTR^{high} myoblasts. (B) Bioinformatic representation of the pathway, including the muscle proteins detected in the array. Gene expression is shown as a gradient with colours from yellow to red, corresponding to increase values from 1.7 to 4.0, respectively. (C) Percentages of cells expressing dysferlin or myogenin in p75NTR positive or negative myoblasts. The experiments presented were carried out in triplicate and repeated at least three times in two primary cell lines. (D-E) Triple immunofluorescence for p75NTR, CD56 and dysferlin (D) or myogenin (E) on an adult control muscle. Enlargement scale 3.5 μm. **P<0.01.

FIG. 5. p75NTR regulates the induction and maintenance of dystrophin.

(A) Percentage of positive myotubes for dystrophin, β-dystroglycan and dysferlin after silencing in the precursor cells. Dystrophin transcript levels (B) and percentage of myotubes positive to the TUNEL (C) in myotubes silenced with siRNA for p75NTR or with control siRNA exposed to IL-1. The myotubes were treated the same day with siRNA and IL-1 and were analysed after two or more days. (D) Immunofluorescence for dystrophin, TUNEL and DAPI in myotubes treated with IL-1 after silencing with siRNA for p75NTR or with control siRNA. Enlargement scale 30 μm. The experiments presented were carried out in triplicate and repeated at least three times. *P<0.05, **P<0.01, ns not significant.

FIG. 6. Induction of p75NTR in mature myofibres in inflamed muscle.

Immunohistochemistry (A-F) and transcript levels (G) for p75NTR in healthy and diseased skeletal muscle. Representative experiments for each group are shown in A-F. In G, the black bars show the average values for each group, each circle represents a separate sample. (H) Electronic microscopy for p75NTR in inflamed muscle. The arrows show immunoreactivity on the cell membrane. (I) Correlation between immunoreactivity for p75NTR in mature myofibres (CD56 negative) and degree of regeneration, measured as the number of CD56 positive myofibres/area. (J-K) Modulation of p75NTR in myotubes after exposure to IL-1. The transcript (J) and protein (K) levels were measured after 24 and 72 hours of stimulation, respectively. Enlargement scale 100 μm in A-F and 1 μm in H. The experiments presented were carried out in triplicate and repeated at least three times. **P<0.01, ***P<0.001.

### BIBLIOGRAPHY

### Baron P, Scarpini E, Meola G, Santilli I, Conti G, Pleasure D, et al.

Expression of the low-affinity NGF receptor during human muscle development, regeneration, and in tissue culture. Muscle Nerve 1994; 17: 276-84.
Bibel M, Barde Y A. Neurotrophins: Key regulators of cell fate and cell shape in the vertebrate nervous system. Genes Dev 2000; 14: 2919-37.
Chevrel G, Hohlfeld R, Sendtner M. The role of neurotrophins in muscle under physiological and pathological conditions. Muscle Nerve 2006; 33: 462-76.
Dalakas M C, Hohlfeld R. Polymyositis and dermatomyositis. Lancet 2003; 362: 971-82.
Dechant G, Barde Y A. The neurotrophin receptor p75(NTR): Novel functions and implications for diseases of the nervous system. Nat Neurosci 2002; 5: 1131-6.
Dennis G, Jr, Sherman B T, Hosack D A, Yang J, Gao W, Lane H C, et al. DAVID: Database for annotation, visualization, and integrated discovery. Genome Biot 2003; 4: P3.
Deponti D, Buono R, Catanzaro G, De Palma C, Longhi R, Meneveri R, et al. The low-affinity receptor for neurotrophins p75NTR plays a key role for satellite cell function in muscle repair acting via RhoA. Mol Biol Cell 2009; 20: 3620-7.
Erck C, Meisinger C, Grothe C, Seidl K. Regulation of nerve growth factor and its low-affinity receptor (p75NTR) during myogenic differentiation. J Cell Physiol 1998; 176: 22-31.
Gonzalez M, Ruggiero F P, Chang Q, Shi Y J, Rich M M, Kraner S, et al. Disruption of trkb-mediated signaling induces disassembly of postsynaptic receptor clusters at neuromuscular junctions. Neuron 1999; 24: 567-83.
McKay S E, Garner A, Caldero J, Tucker R P, Large T, Oppenheim R W. The expression of trkB and p75 and the role of BDNF in the developing neuromuscular system of the chick embryo. Development 1996; 122: 715-24.
Mousavi K, Jasmin B J. BDNF is expressed in skeletal muscle satellite cells and inhibits myogenic differentiation. J Neurosci 2006; 26: 5739-49.
Needham M, Mastaglia F L. Inclusion body myositis: Current pathogenetic concepts and diagnostic and therapeutic approaches. Lancet Neurol 2007; 6: 620-31.
Raivich G, Zimmermann A, Sutter A. The spatial and temporal pattern of beta NGF receptor expression in the developing chick embryo. EMBO J 1985; 4: 637-44.
Rende M, Brizi E, Conner J, Treves S, Censier K, Provenzano C, et al. Nerve growth factor (NGF) influences differentiation and proliferation of myogenic cells in vitro via TrKA. Int J Dev Neurosci 2000; 18: 869-85.
Seidl K, Erck C, Buchberger A. Evidence for the participation of nerve growth factor and its low-affinity receptor (p75NTR) in the regulation of the myogenic program. J Cell Physiol 1998; 176: 10-21.
Sheard P W, Musaad K, Duxson M J. Distribution of neurotrophin receptors in the mouse neuromuscular system. Int J Dev Biol 2002; 46: 569-75.
van Iersel M P, Kelder T, Pico A R, Hanspers K, Coort S, Conklin B R, et al. Presenting and exploring biological pathways with PathVisio. BMC Bioinformatics 2008; 9: 399.
Wang X H, Poo M M. Potentiation of developing synapses by postsynaptic release of neurotrophin-4. Neuron 1997; 19: 825-35.
Wheeler E F, Bothwell M. Spatiotemporal patterns of expression of NGF and the low-affinity NGF receptor in rat embryos suggest functional roles in tissue morphogenesis and myogenesis. J Neurosci 1992; 12: 930-45.
Xie K, Wang T, Olafsson P, Mizuno K, Lu B. Activity-dependent expression of NT-3 in muscle cells in culture: Implications in the development of neuromuscular junctions. J Neurosci 1997; 17: 2947-58.

### Yamamoto M, Sobue G, Yamamoto K, Terao 8, Mitsuma T.

Expression of mRNAs for neurotrophic factors (NGF, BDNF, NT-3, and GDNF) and their receptors (p75NGFR, trkA, trkB, and trkC) in the adult human peripheral nervous system and nonneural tissues. Neurochem Res 1996; 21: 929-38.
Ian Q, Johnson E M, Jr. An immunohistochemical study of the nerve growth factor receptor in developing rats. J Neurosci 1988; 8: 3481-98.
Yano H, Chao M V. Mechanisms of neurotrophin receptor vesicular transport. J Neurobiol 2004; 58: 244-57.

1. Marker for human or animal muscle high differentiating power stem cells consisting in the receptor recorded as P08138, TNR16_HUMAN in UniProtKB database, expressed in human or animal skeletal muscle cells.

2-3. (canceled)

4. Method for the identification of human or animal muscle high differentiating power stem cells consisting in determination of cellular expression of the receptor recorded as P08138, TNR16_HUMAN in UniProtKB database.

5-9. (canceled)

10. Method for the control, regulation and induction of dystrophin production in human or animal muscle by inducing or stimulating cellular expression of the receptor recorded as P08138, TNR16_HUMAN in UniProtKB database.

11-15. (canceled)
